(19) United States
(12) Patent Application Publication
(10) Pub. No.: US 2005/0053585 A1
(43) Pub. Date: Mar. 10, 2005

(54) Cell constructs cultured in vitro, preparation and uses

(76) Inventors: Annie Black, Marseille (FR); Jean-Louis Romette, Marseille (FR)

Correspondence Address: NIXON & VANDERHYE, PC, 1100 N GLEBE ROAD, 8TH FLOOR, ARLINGTON, VA, 22201-4714, US

(21) Appl. No.: 10/498,264

(22) PCT Filed: Dec. 13, 2002
(86) PCT No.: PCT/FR02/04360

(30) Foreign Application Priority Data
Dec. 14, 2001 (FR) FR 01 16265

(51) Int. Cl. A61K 45/00; C12N 5/08

(52) U.S. Cl. 424/93.7; 435/366

(57) ABSTRACT

The present invention relates to a cell construct cultured in vitro characterized in that it comprises (i) animal cells cultured in vitro in conditions ensuring formation of a three-dimensional tissue structure and (ii) an endogenous extracellular matrix in which said cells are imprisoned, and in that it comprises, among said cells, cells having a mineralization and/or ossification capacity. The invention is particularly useful in the fields of grafts and implantology (tissue reconstruction or repair), as well as in the pharmaceutical industry, for research on tissue and analysis of the toxic or beneficial profile of molecules capable of being used in pharmaceutics development and/or pharmaceutical compositions.

### INTRODUCTION AND STATE OF THE ART

The present invention relates to the technical fields of biology, biotechnology, toxicology, pharmacology and medicine. Its applications concern in particular the fields of human and veterinary health. More particularly, the invention describes novel methods for the culture and reconstruction of human or animal tissues in vivo or ex vivo, typically from the cells composing them. Said methods preserve the capacities of the cells used to differentiate all while orienting this differentiation in the desired direction. The invention also concerns the cell constructs and tissues so reconstituted, and the numerous uses that can be made of them. It further provides tools and kits for practicing said methods.

The invention is particularly useful in the fields of grafts and implantology (tissue reconstruction or repair), as well as in the pharmaceutical industry, for research on tissue and analysis of the toxic or beneficial profile of molecules capable of being used in pharmaceutics development and/or pharmaceutical compositions.

Tissue reconstruction combines the techniques of bioengineering with the principles governing the life sciences so as to understand the structural and functional interrelationships of normal and pathological tissues in mammals. One of the sought-after goals is to produce biological substitutes to test, restore, maintain or improve biological functions in vitro or in vivo, and to produce in vitro tissues or cell constructs resembling as closely as possible to the native tissues the reconstruction of which is desired.

The use of cells from higher eukaryotes for experimental research (genetic studies, toxicity studies, and the like), or for pharmacological or therapeutic purposes (cell therapy, tissue repair, and the like) has become increasingly widespread. To this end, different methods for obtaining cells or tissues have been developed, in particular for preparing primary cell cultures, based principally on sampling and ex vivo treatment of a biopsy.

Previously described methods of tissue reconstruction (for example of vessels) are based mainly on the use of a porous membrane or an exogenous synthetic scaffold, designed to support and enable the culture of cells from the tissue to be reconstructed. In addition, although tissue cultures have been carried out in the absence of a synthetic matrix or porous membrane, they are limited mainly to specific tissues and do not have the characteristics suited to a direct therapeutic use.

### GENERAL DESCRIPTION OF THE INVENTION

The problem that the present invention proposes to resolve is that of obtaining a functional cell construct capable of biologically integrating in the tissues of a recipient host subject, with the help of the resources of the cells of said construct and the properties of said construct. In the spirit of the invention, particularly advantageous tissues are tissues capable of exhibiting functional behavior when grafted into a host, that is to say, of durably incorporating and integrating in said host and/or being progressively remodeled by the cells of the host, with the aim of compensating, repairing or restoring deficient properties.

A first particular aspect of the invention concerns reconstructed tissues or cell constructs comprising cells having a mineralization and/or ossification capacity.

Another aspect of the invention relates to reconstructed cell or tissues constructs capable of incorporating or integrating durably in a host and comprising zones of mineralization and/or ossification.

The tissues or cell constructs of the invention may be produced from different cell types, alone or in combination, in particular from ligament, tooth, bone, tendon cells, and comprising an extracellular matrix in which said cells are imprisoned.

A specific aspect of the invention is based on a tissue reconstructed from ligament cells, particularly periodontal ligament.

Another particular aspect of the invention is to provide reconstructed tissues with high thickness, typically greater than or equal to approximately 100 μm, particularly in which the cells and the matrix are functionally organized.

The present invention relates in general to cellular tissues (or constructs) reconstructed from animal cells, preferably mammals, for example from human cells, cultured in vitro without use of a synthetic scaffold or matrix intended to support the tissue. The cell constructs may thus be advantageously prepared solely from animal cells, preferably mammalian, for example human cells, and endogenous extracellular matrix components (that is to say, produced by said cells).

Another aspect of the invention is based on a therapeutic composition comprising a reconstructed cellular tissue such as defined hereinabove. In particular, the invention provides cellular dressings (or bioactive dressings) comprising a reconstructed tissue or cell aggregate such as defined hereinabove, and designed to be implanted in a subject.

The invention also concerns methods of treatment or tissue repair, comprising preparing a reconstructed tissue and implanting it in a subject, in an autologous, allogeneic or xenogeneic context, preferably in an autologous context.

The invention is useful for treating or repairing tissue defects in subjects, for preparing implants, for performing in vitro or ex vivo studies, and the like.

### DETAILED DESCRIPTION OF THE INVENTION

The present invention concerns cell constructs prepared in vitro, production and uses thereof.

A particular object of the invention more specifically relates to a cell construct wherein it comprises:
(i) animal cells cultured in vitro in conditions ensuring formation of a three-dimensional tissue structure, and
(ii) an endogenous extracellular matrix in which said cells are imprisoned,
   and wherein it comprises, among said cells, cells having a mineralization and/or ossification capacity.

In the spirit of the invention, the term “construct” or “cell construct” designates a cellular tissue or aggregate in a spread-out form. It may be an aggregate or tissue comprising one or more layers of cells stacked on top of each other, typically from 1 to 20 cell layers, preferably from approximately 2 to 15 layers. The cells of the construct are imprisoned in a dense three-dimensional extracellular matrix synthesized by the very cells of the construct, and which gives the inventive construct its structure and support. The term construct generally denotes an artificial tissue construct (that is to say, in particular, produced, cultured or maintained in vitro) comprising one or more layers of cells cultured in vitro, imprisoned in an extracellular matrix produced by said cells, cells of the construct being biologically active, that is, capable of dividing, proliferating, remodeling, secreting biological factors, etc. According to the origin of the cells and the culture conditions, the contruct generally has a polarity, with a basal face and an apical face having different properties.

### Mineralization

A particularly advantageous characteristic of the cell constructs or reconstructed tissues according to the invention is based on the fact that they can comprise cells having conserved a mineralization and/or ossification capacity. The present application indeed shows that it is possible to artificially reconstitute tissues or constructs having mineralization capacity, thereby creating mineralized zones. This aspect of the invention is particularly unexpected and important, since it enables the construction of tissues having biological properties adapted to the reconstitution of defects and/or displaying greatly improved properties of integration in a host.

In the spirit of the invention, cells having mineralization/ossification capacity is understood to mean cells which produce, naturally or after specific stimulation, an enzymatic activity catalyzing the formation and/or accumulation, in the extracellular matrix, of calcium phosphate or calcium carbonate or a mixture of these different ions, typically in the form of particles, deposits, patches, etc. Typically they are cells which produce, in the culture conditions, alkaline phosphatase and/or additional proteins (BMP, BSP, OPN, OCN, etc.), in this manner allowing the production of inorganic phosphate.

The invention shows that the constructs may be produced from explants, in which the cells retain the capacity to produce mineralized zones, in spite of the culture and/or treatment steps performed.

Said mineralization generally leads to formation of a mineralized and/or ossified face of the cell construct, which generally corresponds to that exposed to the culture support. As described in the text that follows, said face is also the richest in collagen. In fact, mineralization takes place within the collagen fibers present in the construct, thus allowing them to be solidly anchored in the mineralization deposits. Moreover, the progenitor cells increase in number in the vicinity of the culture support and facilitate the mineralization initiated by the matrix and the collagen network.

This feature of the invention is particularly important and advantageous for producing cell constructs for repairing structures such as ligaments, tendons, teeth, bones, joints and the like, in so far as the production of a mineralization promotes anchoring of the collagen fibers of the tissue in the mineralized layer and leads to a reconstructed tissue more closely resembling the native tissue, notably a tissue containing a cement.

The mineralizing cells of the inventive constructs are typically mesenchymal cells or precursors thereof. Their mineralization capacity is conserved by the culture conditions and/or treatment of the cells in the construct, and/or due to the specific tissue origin of said cells.

### Cell Composition

As noted earlier, the reconstructed tissues or constructs according to the invention advantageously comprise animal cells, in particular mammalian cells, preferably human cells. They may be undifferentiated stem cells or cells derived from mature tissues, or a combination of such cells. If the cells are sampled from a mature tissue, they may be any type of cell able to produce extracellular matrix and/or conserve or acquire a mineralization and/or ossification capacity particularly under the influence of external factors. The cells involved in tissue reconstruction may thus come from different cell populations and so form a heterogeneous population.

For example they may be cells derived from mature tissues like muscle, bone, tooth, cartilage, tendon or ligament, in particular tooth and ligament, particularly periodontal ligament or anterior cruciate ligament. Preferably, when the cells are derived from periodontium, they comprise, at least in the initial cultures, cells from the periodontal ligament, cement and dentin cells, namely, mainly fibroblasts (approximately 80%). Cells derived from the periodontal ligament typically comprise fibroblasts, epithelial cells, cementoblasts, osteoblasts and/or progenitors of these different cell types. When they are derived from tooth, they comprise odontoblasts, ameloblasts, pulp cells, epithelial cells, cementoblasts and/or progenitors of these different cell types. It may also be a matter of a sample containing a majority of undifferentiated stem cells the differentiation of which will be oriented by suitable culture methods. The cells may also be chondrocytes or osteoblasts. Said cells may originate from mesenchymal stem cells and/or cells isolated from a bone marrow extract. However, the presence for example of cells of the osteoblast or chondroblast type or precursors of said cells of the pre-osteoblast or pre-chondroblast type or else cells harvested from bone marrow is not necessary to obtain a construct according to the invention presenting in particular zones of mineralization and/or ossification.

The tissue according to the invention may be composed of a single cell population or a combination or mixture of several cell types (or populations), uniformly distributed or not within the tissue so as to mimic the cellular composition and structure of the native tissues. If different cell types are present, it is preferable that at least one of said cell types be capable of mineralization and/or ossification. The cell type(s) not possessing a mineralization capacity may further be stimulated or transformed in such a way as to induce mineralization and/or ossification.

It is also possible to induce the cells of the construct (for example fibroblasts) to express compounds that are naturally absent, for instance by exposing them to a chemical substance, by applying a physical stimulus or else by using transgenic methods.

The different cell populations may be associated at the start of the culture period, for example in a given ratio, or else successively, for example by combining several constructs.

A particular object of the invention is a cell construct such as defined hereinabove, produced from (or comprising) ligament cells cultured in vitro, in particular from periodontal or anterior cruciate ligament. The invention shows that artificial mineralized tissues may be produced from this type of cell, which may be remodeled to form thick constructs, and which exhibit a functional biological organization.

### Endogenous extracellular matrix

As indicated earlier, the tissues or constructs according to the invention comprise cells which are surrounded by or embedded or imprisoned in an endogenous extracellular matrix, that is to say produced by the cultured cells. The extracellular matrix typically (and principally) comprises a network of collagen fibers organized notably into fibrils, sulfated proteoglycans, potentially involved in in vivo regulation of fibril diameter, as well as simple or sulfated glycosaminoglycans (GAG). Simple GAG typically include hyaluronic acid (HA) and/or fibronectin and, among the sulfated GAG, di-hyaluronic acid, di-chondroitin-0-sulfate, di-chondroitin-4-sulfate, di-chondroitin-6-sulfate, di-chondroitin-4,6-sulfate, di-chondroitin-4-sulfate-UA-2S and/or di-chondroitin-6-sulfate-UA-2S are particular examples. The extracellular matrix may additionally comprise decorin, such as biglycan or versican, as well as tenascin, an extracellular glycoprotein of the matrix found in particular in mesenchyme or in tissues undergoing repair. It appears that the cultured cells gradually fill the space separating them with a loose matrix rich in type III collagen and fibronectin and then remodel this matrix with type I collagen to make it denser.

It is not necessary for the exact composition of the extracellular matrix to be known, in order to practice the invention. Typically, the construct comprises cells having the ability to synthesize an endogenous extracellular matrix, the composition of which is similar to that found in the original tissue, unless intentionally modified (for example, by incorporation of synthetic materials, genetic modification of the cells, etc.).

In a particular embodiment, the present invention concerns a construct such as defined hereinabove comprising a basal layer and an apical layer relative to the culture support, said basal layer being rich in collagen and also comprising fibroblasts and progenitor cells ensuring mineralization and/or ossification and said apical layer being less rich is collagen than said basal layer, and typically comprising more proliferative cells.

### Dimensions of the construct

The inventive constructs may be of variable dimensions, in area as well as in thickness. Moreover, they may have or adopt different forms. The dimensions and the form may be adapted by those skilled in the art or by the user, according to the desired uses.

A particularly advantageous characteristic of the inventive constructs or tissues is their considerable thickness, typically comprised between 30 and 120 microns, preferably between 50 and 120 μm, even more preferably greater than or equal to approximately 100 μm. In fact, the present application shows that it is possible to produce cell constructs in vitro having a high thickness, in which the cells are capable of organizing, differentiating and proliferating, to create a. functional polarity in the construct without causing any particular cellular necrosis.

Said characteristic is advantageous because it provides a reconstructed tissue in which the cells organize and develop advantageous biological properties. For instance, due to the increased thickness of the tissue, the cells behave differently. In particular, the global endogenous diffusion of substances within the tissue, and therefore in the cell environment, is diminished, which increases the potential effect of the cells and leads to their reorganization and remodeling. This organization also promotes mineralization, polarization of the tissue, expression of a proliferative layer on the apical face, support of the construct and/or its integration in a subject.

The thickness of the construct may be modified in different ways. Preferably, it is done artificially, for example by folding or rolling a thinner construct upon itself, so as to increase its thickness by merging and remodeling of the different cell layers. In fact, the invention shows that it is possible to compact a first tissue construct comprising approximately 3 cell layers obtained. by culture, and that said compacting (for example, folding, rolling, etc.) provides a tissue in which the cells organize, differentiate, and develop advantageous biological properties. The invention also shows that it is possible to cut a tissue or construct into pieces which may then be folded or rolled on themselves. The thickness may also be increased artificially by detaching the edges of the construct, which causes its retraction and compacting of the cells.

In this respect, a particular object of the invention is also based on a cell construct, characterized in that it comprises (i) animal cells cultured in vitro in conditions ensuring formation of a three-dimensional tissue structure and (ii) an endogenous extracellular matrix in which said cells are embedded (or imprisoned), and wherein it has a thickness greater than or equal to approximately 100 μm.

More particularly, the construct contains or is produced from cells having a mineralization capacity, and/or contains or is produced from ligament, tendon, tooth or bone cells.

A more particular object of the invention is thus based on a cell construct, characterized in that it comprises (i) cells sampled from ligament, tendon, tooth and/or bone of mammal(s), cultured in vitro in conditions ensuring formation of a three-dimensional tissue structure, and (ii) an endogenous extracellular matrix in which said cells are embedded (or imprisoned), and wherein it has a thickness greater than or equal to approximately 100 μm.

Another more preferred object of the invention is thus based on a cell construct, wherein it comprises (i) cells sampled from ligament, tendon, tooth and/or bone of mammal(s), cultured in vitro in conditions ensuring formation of a three-dimensional tissue structure and (ii) an endogenous extracellular matrix in which said cells are embedded (or imprisoned), wherein it has a thickness greater than or equal to approximately 100 μm and wherein it contains cells having a mineralization capacity and/or mineralized zones.

Typically, the construct comprises a basal layer (face) and an apical layer (face), the basal layer being rich in collagen and mineralized zone.

As indicated, the area of the construct or tissue may be adapted by those skilled in the art by varying the quantity of seeded cells, the culture time, the size of the culture container, etc. Furthermore, as indicated, the construct may be cut into pieces the shape of which is adapted to the subsequent use of the construct, for example rectangular, square, circular, etc. and an area also adapted to said use, for example comprised between 1 and 20 cm², preferably between 1 and 15 cm², even more preferably between 2 and 10 cm².

### Preparation

The tissues or constructs of the invention may be produced in different ways. According to a particularly preferred embodiment, which is also an object of the present application, the cell constructs are prepared by a method typically comprising:
a) culturing the cells of interest in conditions adapted to the synthesis of an extracellular matrix and to formation of a construct comprising one or more cell layers imprisoned in the neosynthesized endogenous extracellular matrix, and
b) recovering the construct.

Said method may also comprise, prior to steps a) and b), the following steps a′) and b′):
a′) extracting the cells of interest from one or more tissues, by any suitable method, for example by enzymatic digestion, mechanical treatment, explant, etc., and
b′) amplifying said cells harvested in step a′) in a suitable medium, typically in the presence of ascorbic acid or a derivative thereof.

The method may also comprise another step c) of recovering the cell construct (for example by detachment) and a step d) of artificially increasing the thickness of said construct, notably by folding (for example by successive folds), rolling the construct on itself or retracting the construct.

In step a′), the cells may be extracted from the chosen tissue by any suitable method, for example by enzymatic digestion (eg., collagenase) or through the use of an explant technique known to the man skilled in the art. It may be done by mechanical dissection (eg., with a scalpel), enzymatic wash, concentration of the cells, mechanical cutting (eg., scissors), and the like. The cells so harvested are generally composed of a population of cells having different natures, for example mature cells and progenitor cells. Said cells are typically representative of the composition of the tissue or construct to be reconstructed.

The cells so obtained are then suspended in the culture medium, so as to carry out step b′) of amplification (or expansion or proliferation). This step aims principally to increase the number of cells so as to facilitate constitution of the tissue. It may be carried out for different periods of time, for example from 1 to 10 days, or longer, according to the cell type, the amount of available cells, etc. The culture medium used is typically a basic culture medium such as described hereinbelow (possibly supplemented with ascorbic acid or a similar molecule), which may be changed 2 or 3 times a week according to the state of cell proliferation. At the end of this step, when the cells have reached confluence or approximately 50% confluence, the culture is treated to detach adherent cells, for example by using trypsin. According to the cell type or degree of confluence, the period of time in the incubator which follows and enables detachment of the tissue, will be more or less long. For example it will be 3 minutes for endothelial cells, 5 minutes for fibroblasts, 8 minutes for keratinocytes, etc. (see examples). During this phase, the cells produce only a small amount of extracellular matrix.

The amplified cells so obtained are then used to prepare the construct. To this end, they are seeded in a suitable container and cultured in conditions adapted to synthesis of an extracellular matrix and to formation of a construct comprising one or more layers of cells imprisoned in the neosynthesized endogenous extracellular matrix.

Typically, the cells are seeded (for example in Petri dishes) at a density comprised between about 10³ and 10⁶ cells/cm², preferably between 3.10³ and 5.10⁵ cells/cm², typically between 3,000 and 12,000 cells/cm². In a typical experiment, one thus uses about 5.10⁵ cells per dish of 75 cm², for example.

The cells are thus cultured for a sufficient period of time to permit:
production of a construct having a desired area, composed of one or more cell layers, typically 1 to 3 (approximately 30 μm thick), imprisoned in an endogenous extracellular matrix,
adhesion of the construct to the support,
proliferation of the cells at the surface, and,
preferably, production or initiation of mineralization (or ossification).

Said culture may be maintained for 2 to 6 weeks, optionally with gentle stirring, advantageously with a medium change 1 to 3 times a week.

To ensure formation of an extracellular matrix, the cultures are advantageously carried out in a basic culture medium comprising ascorbic acid or a derivative thereof. The ascorbate is added to promote proline hydroxylation and secretion of procollagen, a soluble collagen precursor, but also because it is an important cofactor for enzymes active during the post-translational phase and it ensures upstream regulation of type I and III collagen synthesis. Ascorbic acid may be replaced by one of its synthetic derivatives or by a nutrient acting on extracellular matrix synthesis. For this reason, the inventive method does not require the use of exogenous matrix.

The basic culture media used are media adapted to the cell type(s) and the tissue or construct to be reconstituted. The medium and culture conditions should stimulate cell growth in the construct and synthesis of extracellular matrix. A medium of defined composition is preferable, that is to say, one that in particular does not comprise animal organ or tissue extract, although the presence of such undefined components is possible if it promotes the production of a construct according to the invention. Synthetic or recombinant functional equivalents known to those skilled in the art may also be added to the culture medium of defined composition. The man skilled in the art may easily determine the basic components required for the culture of animal cells. Among the commercially available media that may be used in the present invention, particular examples are media supplying inorganic salts, an energy source, amino acids and vitamin B such as DMEM (Dulbecco's Modified Eagle's Medium), MEM (Minimal Essential Medium), RPMI 1640, EDMEM (Iscove's Modified Dulbecco's Medium), Ham media (in particular Ham's F-12) or NCTC 109. A preferred basic medium according to the invention is a mixture of DMEM/Ham F12 (50:50 by volume). The medium may contain serum. The basic medium is typically supplemented notably with compounds such as amino acids and in particular proline and glycine which are components of the collagen structure, growth factors, hormones and inorganic salts. Specific culture media useful in the invention are described in application WO96/21003. Other media are described in the examples.

The cultures may be carried out in any suitable device, such as plate, dish, flask, bag, etc. Typically it is a Petri dish or similar device. The cultures are advantageously maintained in an incubator which allows control of temperature, humidity and gas mix. Preferred culture conditions are for example a temperature comprised between approximately 35 and 40° C., preferably approximately 37° C., an atmosphere containing 5 to 10% CO₂ and a relative humidity comprised between approximately 75 and 95%.

The tissue produced in this manner contains cells associated with a dense three-dimensional network of extracellular matrix newly synthesized by said cells. During this phase, and in response to stimulation of the cells by ascorbic acid or a derivative thereof, the cells multiply, stack and produce an extracellular matrix whose composition is essentially identical to that of a natural extracellular matrix. When the cell construct is prepared from cells derived in particular from tendon or ligament, skin or dermis, it thus comprises, at approximately three weeks of culture, a large proportion of collagen concentrated in particular in the half of the construct whose face directly contacts the culture support. It is in this collagen network that mineralization will be initiated.

In an advantageous embodiment of the inventive method, the cells are cultured in conditions ensuring formation or maintenance of the cells' capacity for mineralization (or ossification). Said capacity may be related to the cells used, and favored by culture in the presence of a stimulus which activates or increases mineralization and/or ossification of the cells, notably of basal progenitor cells.

In a particular embodiment, tissue mineralization is achieved in a surprising manner by modifying the culture conditions. Said mineralization concerns the neosynthesized matrix and in particular the matrix constituting the half of the construct in contact with the culture support. Stimulation may be produced by contacting the construct or cells with a particular material or with particles of said material, by adding differentiation factor(s), conditioned medium, synthetic substances or else by adding natural substances such as coral or by mechanical stimulation. Examples of useable mineralizing materials are notably a support or particles comprising hydroxyapatite, bioactive glass (or bioglass), a mineralized collagen substitute, a bone substitute, a ceramic, particularly zirconium-based, or coral, or any material (for example a composite material) whose surface is coated, at least partly, with one of said components. For instance, the mineralizing material may also be produced from an inert material bio-activated by surface grafting of substances able to induce mineralization of the construct. Examples of useful substances or factors include for instance adjuvants added to the culture medium and reinforcing the mineralization stimulant effect created by the selected support (cytokine, dexamethasone, beta-glycerophosphate, beta-aminopropionitrile).

In an advantageous embodiment, the invention therefore comprises a method for preparing a mineralized or mineralizing cell construct, comprising a step of contacting the cells with a stimulus such as a mineralizing support or material, particles of said material, differentiation factors, cytokines, conditioned medium, a synthetic substance or a natural substance, stimulating mineralization or the mineralization capacity of the cells. Preferably the stimulus is induced by a mineralizing support or material, advantageously comprising hydroxyapatite or bioglass. Eventually, hydration of the bioglass leads to its resorption, and this type of support is therefore especially adapted and advantageous when the tissue to be reconstructed is intended for transplantation, since ultimately only the transplant remains in the recipient host.

A particular object of the invention is therefore based on a method for preparing a reconstructed cellular tissue or construct, comprising culturing cells in vitro in conditions ensuring synthesis of extracellular matrix and mineralization. In an advantageous manner it is a culture in the presence of a stimulus, such as a mineralizing treatment, agent or support, advantageously a culture on a mineralizing support or in the presence of mineralizing particles. The production of a construct according to the invention having zones of mineralization does not require addition of growth factor, such as TGF-βl for example.

A particular object of the invention also relates to a construct or cellular tissue, characterized in that it may be obtained by a method comprising culturing cells in vitro, in the absence of synthetic matrix or scaffold, in conditions ensuring formation of a construct comprising one or more cell layers imprisoned in an endogenous extracellular matrix, and in conditions stimulating mineralization or ossification.

Mineralization is generally carried out or initiated at the same time as the step of construct formation and extracellular matrix synthesis. However, mineralization generally continues after recovery of the construct and/or its artificial thickening.

A particular object of the invention therefore consists in placing the cultured cells in the presence of mineralizing agents, for instance micro or nano-particles of bioglass or hydroxyapatite, right from the start of the culture. Said agents promote the migration of progenitor cells and stimulate their mineral and/or bone-forming capacity.

Such mineralization and/or ossification results in a particularly advantageous improvement in the integration of the reconstituted tissue or cell construct within the tissues of the recipient host.

Mineralization may be observed by staining with paragon or von Kossa stain for example. As a specific example, periodontal cells seeded at a density of approximately 5,000 cells/cm² may thus be cultured for approximately 3 weeks, in the presence of hydroxyapatite microparticles (see FIG. 5). The cultured cells recognize the stimulus and produce specific enzymes of the alkaline phosphatase type. Phosphated substrates like β-glycerophosphate for example will be hydrolyzed. They thus release phosphates able to induce partial crystallization of the tissue by complexing with calcium ions to form calcium phosphate.

As noted, the tissues or constructs may be produced in any suitable device, such as a plate, dish, flask, bag, etc. Typically it is a Petri dish or similar device. Preferably, the preparation is carried out in the absence of synthetic three-dimensional matrix or scaffold.

A particular object of the invention is based on a method for preparing a reconstituted construct or tissue, comprising in vitro culture of cells from periodontal or cruciate ligament, in conditions ensuring the synthesis of extracellular matrix.

Once the construct is produced, it may be recovered from the support by any known technique, typically by detachment. As a matter of fact, one of the characteristics of the invention is the ease of recovering the reconstituted cell construct by simply detaching said contruct from its culture support.

Moreover, in supplemental step d), the construct or tissue so recovered may be treated (eg., compacted) in view of increasing its thickness, typically by folding, rolling, aggregating, etc. Generally, before being artificially increased, the thickness of the cell construct corresponds to about 2 or 3 layers of cells stacked on top of each other, or approximately 30 μm. In fact, the cells are generally kept in culture until they have secreted a sufficient amount of matrix to provide minimal support of the cell construct. It is then possible to detach the cell construct from its culture support without tearing it, and to produce a cell construct having an increased thickness, for example increased two to five-fold relative to a thin cultured construct.

A particular object of the invention also relates to cellular constructs or tissues, characterized in that they may be produced by a method comprising culturing cells in vitro, in the absence of synthetic matrix or scaffold, in conditions ensuring formation of a construct comprising one or more cell layers imprisoned in an endogenous extracellular matrix, and a step of detaching the construct from its culture support, folding or rolling the construct upon itself, so as to produce a reconstructed tissue having increased thickness.

Another particular object of the invention also relates to cellular tissues or constructs, characterized in that they may be produced by a method comprising culturing periodontal or anterior cruciate ligament cells in vitro, in the absence of synthetic matrix or scaffold, in conditions ensuring formation of a construct comprising one or more cell layers imprisoned in an endogenous extracellular matrix, and, preferably, a step of detaching the construct from its culture support, folding or rolling the construct upon itself, so as to produce a reconstructed tissue having increased thickness.

The inventive constructs may be maintained in culture for different periods of time, typically in the presence of medium, to promote cellular reorganization, continued mineralization, etc. The constructs or tissues may then be used for clinical (transplant, implant) or pharmaceutical applications.

### Storage/Maintenance

The inventive constructs or tissues may be stored in any suitable device, in the presence of culture medium, nutrient medium or isotonic saline solutions. Typically, the tissues are stored or maintained in dishes, tubes, flasks, ampoules, bags, etc., preferably in the presence of a large quantity of medium. The tissues may be used extemporaneously or stored, preferably in the cold.

### Uses

The constructs or tissues described hereinabove may be used in the field of grafts or implantology as well as in the pharmaceutical industry, for research on tissues and analysis of the toxic or beneficial profile of molecules, particularly candidate medicaments.

A tissue or construct of the invention may thus be used in the field of grafts and implantology. In such case, the tissue or construct preferably has a high thickness, typically approximately 100 μm. Advantageously, then, it is a compacted construct, eg., folded or rolled on itself so as to increase its thickness by merging and remodeling of the different cell layers as described hereinabove, or simply detached from the support to form an aggregate of cells.

For use in tissue repair, the inventive constructs may be used as is, in the form of cell dressings or “patches”, or to coat or cover all or part of an implant to be introduced in a subject.

The inventive tissue or cell construct may be directly used in a graft. The invention indeed provides for the production of a cellular dressing, comprising principally an aggregate of cells such as defined hereinabove. An object of the invention is therefore based in particular on a pharmaceutical composition comprising a construct or tissue such as described hereinabove. In an advantageous manner, it is a thick or compacted tissue such as described hereinabove. The dressing may be applied directly on a cavity or on a damaged tissue, during simple surgical procedures. For example, if the tissue is reconstructed from periodontal cells, the inventive dressing may be used for treatment of periodontal pockets. If it is matter of ligament cells, it is possible to reconstruct or repair a ligament such as the anterior cruciate ligament for example. If the tissue is reconstructed from chondrocytes having conserved their mineralization and/or ossification capacity, a graft may also be directly envisioned at the affected site in the recipient host.

The invention therefore also provides a method of tissue repair or treatment, comprising preparing a reconstructed tissue by in vitro culture as described hereinabove, and injecting said tissue, possibly after packaging in any pharmaceutically acceptable solution or vehicle. In this context, the tissue may optionally be associated with an exogenous matrix, such as collagen.

In another embodiment, the inventive tissue or construct is used to prepare an implant, particularly to cover or coat all or part of an implant. It may be a dental implant (see example 4 relating to “Ligaplant”), a ligamental implant, a bone or cartilage prosthesis, etc. In such case, the construct produced is typically arranged or rolled around the structure of the implant.

A dental implant may for example comprise a coronary part on which will be attached the crown of the tooth to be replaced and a radicular part corresponding to the root of said tooth and around which it is possible to roll a cell construct according to the invention. The present invention therefore concerns a method for preparing an implant by means of a construct such as described hereinabove, comprising rolling said construct at the surface of the implant and culturing said implant associated with said construct in conditions allowing maintenance or stimulation of cell proliferation and/or differentiation all while promoting the merging and remodeling of the different cell layers of said construct at the implant surface.

In particular, said construct may be reconstructed from periodontal cells cultured for approximately three weeks in the conditions indicated hereinabove (also see FIG. 4). The mineralized face of the cell construct which corresponds to that exposed to the culture support is then directly exposed to the implant, in an amplification medium and in the presence of ascorbic acid. That face of the cell construct then enables the entire construct to adhere to the implant in a stable, strong and particularly advantageous manner, in particular thanks to progenitor cells which will increase in number around said implant and facilitate the mineralization initiated by the matrix and the collagen network. The structure so reconstructed is then very similar to the tooth's natural cement. As far as the amplification medium is concerned, it facilitates the merging and remodeling of the different cell layers of the construct, in so far as no nutritional restrictions are imposed. The mineralized tissue according to the invention thus globally improves integration of the implant in the recipient alveolar site.

A construct according to the invention may therefore be used to prepare a dental or ligamental implant for example.

Another object of the invention relates to the use in the pharmaceutical industry of a cell tissue or construct according to the invention, for research on tissue and analysis of the toxic or beneficial profile of molecules. In a particular embodiment, the reconstructed tissues of the invention (or a part thereof) are contacted with one or more test compounds, and the effect of said test compound is determined, for example so as to characterize the cellular reactions to said test compound(s).

The test compound may be quite varied in nature. For instance, it may be an isolated compound or a mixture of substances. The compound may be chemical or biological in nature. In particular it may be a peptide, polypeptide, nucleic acid, lipid, carbohydrate, a chemical molecule, plant extracts, combinatorial libraries, etc. The test compound may also be a treatment applied to tissues (irradiation, UV, etc.).

To carry out the inventive method, the test compound may be applied at different concentrations, chosen by the user.

The present invention therefore has as its object the use of a reconstructed tissue such as defined hereinabove, for evaluating (in vitro or ex vivo) the biological and/or toxic properties of a test compound, particularly molecules intended for a therapeutic use.

It also has as object the use of a reconstructed tissue such as defined hereinabove, for screening molecules intended for therapeutic use.

The present application also has as object kits for practicing the methods of preparation and study and also for use of the reconstructed tissues such as described hereinabove. Such kits advantageously comprise a container and/or culture reagents, and/or a tissue construct such as defined hereinabove.

Other aspects and advantages of the invention will be described in more details in the following examples, which are given for purposes of illustration and not by way of limitation.

### LEGENDS OF FIGURES

FIG. 1: Cells extracted from periodontal ligament seeded at a density of 5,000 cells/cm² at J2 (A) after 3 weeks of culture (B). The cells multiply, stack and synthesize extracellular matrix.

FIG. 2: Gross appearance of a construct after 3 weeks of static culture in a Petri dish 100 mm in diameter.

FIG. 3: Mineralized tissue. Paragon staining highlights the mineral deposits.

FIG. 4: Dental implant and its association with a tissue according to the invention.

FIG. 5: Tissue mineralized by addition of hydroxyapatite for treatment of periodontal pockets. Periodontal cells were cultured in the presence of hydroxyapatite microparticles for 3 weeks. The newly synthesized collagen matrix contains mineralization deposits (von Kossa stain).

FIG. 6: Histological section (magnification×100) of a construct prepared from periodontal ligament cells (cells from the cemento-amelar junction) cultured at the surface of a hydroxyapatite support, according to the method of the invention, and implanted under the skin of an athymic mouse (Swiss nu/nu mouse) for 12 weeks. The sample was paraffin-embedded after demineralization and staining was with Masson trichrome.

Cell nuclei appear as dark spots in the “cell” part. Collagen fibers (Sharpey fibers) and proteins of the mineralized matrix in contact with the hydroxyapatite support correspond to the porous mass labeled “HA”.

The resulting tissue structure is similar to that of a fibrillar cellular cement as found in a normal tooth.

FIG. 7: This figure shows a tissue composed of a single sheet, immunohistochemically labeled to detect type V collagen. The light gray color represents the counter-stain and the dark gray color is the labeled type V collagen.

FIG. 8: This figure shows a tissue identical to that of FIG. 7 after being folded or rolled. The labeling profile shows that remodeling has occurred (without conserving the polarity of each fold in the tissue). The light gray color represents the counter-stain and the dark gray color is the labeled type V collagen.

### EXAMPLES

Materials and Methods

The mediums and/or reagents used (eg., vitamin C, ascorbic acid 2-phosphate, collagenase, dexamethasone, etc.) were generally of commercial origin or commercially available. Generally, these reagents were sterilized before use.

Collagenase A (clostridiopeptidase A) is thus a bacterial protease produced by Clostridium histolyticum. This protease shows specificity for X-Gly bonds of Pro-X-Gly-Pro sequences (X representing a neutral amino acid) found in collagen triple helices. Collagenase is not inhibited by serum but by EDTA and is activated by calcium. Its optimal pH is between 6 and 8. In freeze-dried form, it is stable between +2 and +8° C. and in solution between −15 and −25° C. Collagenase can be reconstituted in buffered solutions, such as PBS, HBSS or DMEM/F12, which contain calcium.

Dexamethasone is a glucocorticoid with a molecular weight of 392.5, which increases the level of alkaline phosphatase. It was prepared from the commercial source (Sigma D-2915).

β-glycerophosphate, with a molecular weight of 216, is hydrolyzed by alkaline phosphatase to phosphate ions, required for efficient mineralization. It was prepared from the commercial source (Sigma G-9891).

For preparation and control of the media, aliquots of the reagents were thawed in a water-bath and decontaminated, before being placed under the laminar flow hood. They were added to liquid DMEM/F12 medium using sterile technique.

Antibiotic-free media were prepared 1 to 2 days before use, for control of sterility. They were mixed and controlled. Antibiotics were added immediately before use. Bacteriological test was carried out by conventional methods. Storage was typically between +2 and +8° C.

Synthetic media

| Products | Concentrations |
|---|---|
| DMEM | 1:1 |
| Ham F12 | 1:1 |
| SV supplemented | 10% |
| with Hyclone Iron | |

| | | |
|---|---|---|
| Ascorbic acid 2- | 1 | mM |
| phosphate | | |
| Penicillin | 100 | IU/ml |
| Gentamicin | 20 | μg/ml |
| Amphotericin B | 1 | μg/ml |

Differentiation media

| Products | Concentrations |
|---|---|
| DMEM | 1:1 |
| Ham F12 | 1:1 |
| SV supplemented with | 10% |
| Hyclone Iron | |

| | | |
|---|---|---|
| Ascorbic acid 2- | 1 | mM |
| phosphate | | |
| β-glycerophosphate | 10 | mM |
| Dexamethasone | 10 | nM |
| Penicillin | 100 | IU/ml |
| Gentamicin | 20 | μg/ml |
| Amphotericin B | 1 | μg/ml |

### Example 1

### Extraction and Culture of a Cell Sheet from Cells Sampled from a Dental Root

In this example, the cells were extracted according to the following steps:

The dental tissue specimens were maintained in transport medium until the time of treatment, if possible within 24 hours of receipt. The tissues were then washed 3 times in PBS, in the presence of antibiotics, so as to eliminate excess blood. The roots of the teeth were then placed in Petri dishes 60 mm in diameter for example with about 5 ml of collagenase A (see paragraph describing the media). The teeth were scraped with a scalpel from halfway down the root to its lower extremity, until small fragments of cement and dentin were obtained. The fragments were incubated at 37° C. overnight (between approximately 16 and 20 hours) in a CO₂ incubator. The cells and tissue fragments were placed in a tube, centrifuged and resuspended in culture medium. The suspension of cells and debris was then reseeded in the extraction Petri dish with 4 ml of culture medium. The culture medium was changed 2 or 3 times a week according to the state of proliferation of the cells. The culture was continued for 10 days to approximately 3 weeks before being ready for trypsinization. The Petri dish should preferably be at approximately 50% confluence.

When the cultured cells were preconfluent or confluent, they were treated as follows:

The culture dish was washed with PBS culture, then trypsin-EDTA was added to cover the entire culture (approximately 5 ml per 75 cm²). The flasks were placed in an incubator at 37° C. Depending on the cell type or degree of confluence, the incubation time was more or less long, preferably less than approximately 5 minutes for endothelial cells or fibroblasts, and less than approximately 15 or 10 minutes, for keratinocytes. Cell detachment was monitored and/or controlled by observation, for example under a microscope. The cell suspension was shaken and transferred to a tube, and an aliquot of approximately 50 pi was taken for a cell count. After centrifugation (about 10 minutes at 1200 rpm), the supernatant was discarded and a quantity of culture medium was added so that the suspension would contain 100,000, 1 million or 10 million cells per ml as needed. The final suspension could be prepared at different concentrations as needed.

### Example 2

### Production of the Construct

The tissue produced contains cells associated with a dense three-dimensional network of extracellular matrix newly synthesized by these same cells. Said extracellular matrix was produced in response to stimulation of the cells by ascorbic acid, a synthetic derivative thereof or a nutrient acting on the synthesis of extracellular matrix, without the need to add exogenous matrix.

Said tissue was produced in culture containers, porous or not, of different sizes according to the desired dimensions. Fibroblasts were seeded at a density comprised between 3,000 and 12,000 cells/cm². The culture medium was changed at regular intervals, for instance 3 times a week for 2 to 6 weeks, with or without shaking of the cultures, so as to maintain environmental (physico-chemical) and nutritional conditions favorable to cell growth.

The cells involved in the tissue reconstruction may come from a same cell population or from different cell populations thus forming a heterogeneous population. They may be fibroblasts from the periodontal ligament, cementoblasts, odontoblasts, ameloblasts, pulp cells, fibroblasts from ligaments and tendons, chondrocytes, osteoblasts, mesenchymatous stem cells, bone marrow extracts, or any other type of cell having mineralization capacity particularly under the influence of external factors or a mixture of said cells. However, the presence for example of cells of the osteoblast or chondroblast type or of cell precursors thereof such as pre-osteoblasts or pre-chondroblasts or else cells extracted from bone marrow is not necessary to obtain a construct according to the invention having in particular zones of mineralization.

The tissue may be composed of several cell types not having a mineralization capacity, but which may be stimulated so as to induce said mineralization. For example, they may be fibroblasts from skin and/or muscle cells.

The tissue may be composed of several cell types at once, uniformly distributed or not.

Mineralization of the tissues may be achieved by modifying the culture conditions so as to induce mineralization of the neosynthesized matrix. Said stimulation may be accomplished by contact with a material or with particles of this same material and/or by adding differentiation factors, cytokines, conditioned medium, dexamethasone, beta-glycerophosphate, beta-aminopropionitrile, by adding synthetic substances of the type calcium phosphate, calcium carbonate, hydroxyapatite, bioactive glass, ceramics, natural substances of the coral type but also by mechanical stimulation. Production of a construct according to the invention having mineralization zones does not require addition of a growth factor, such as TGF-β1 for example.

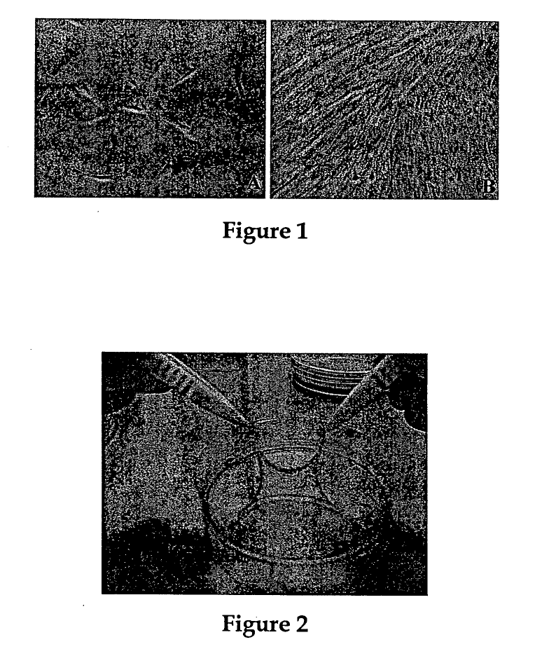

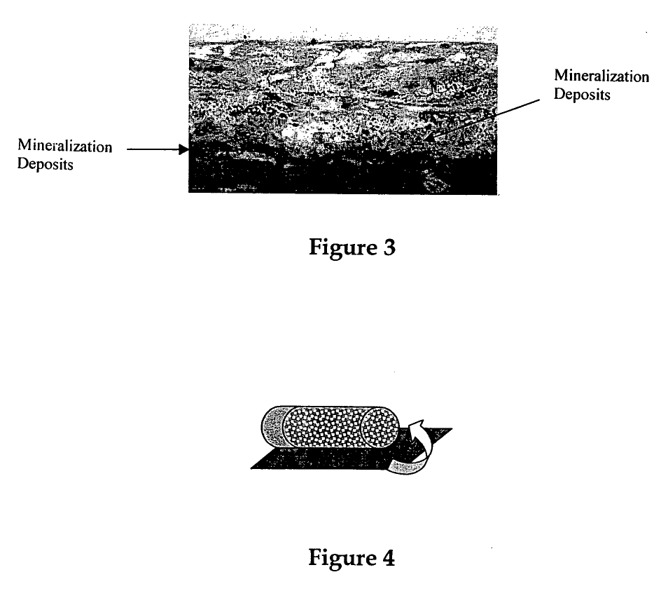

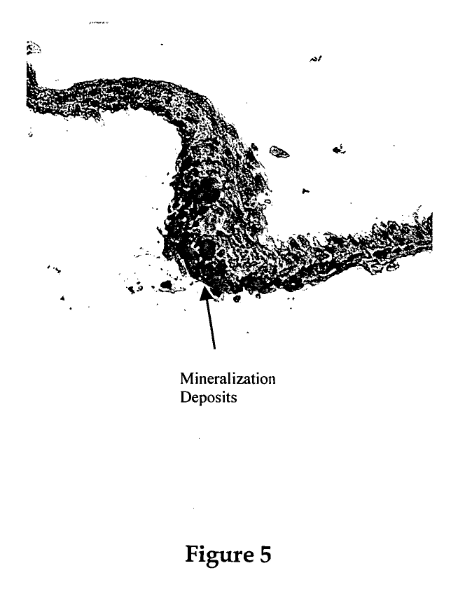

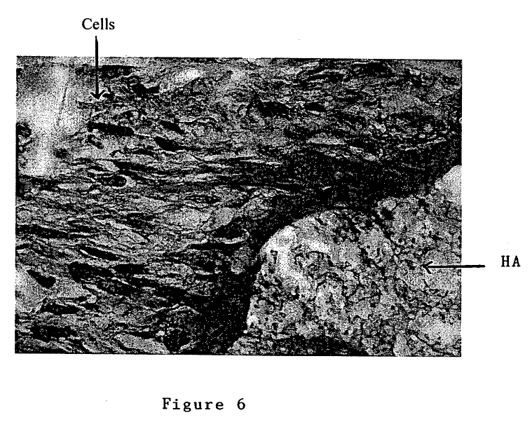

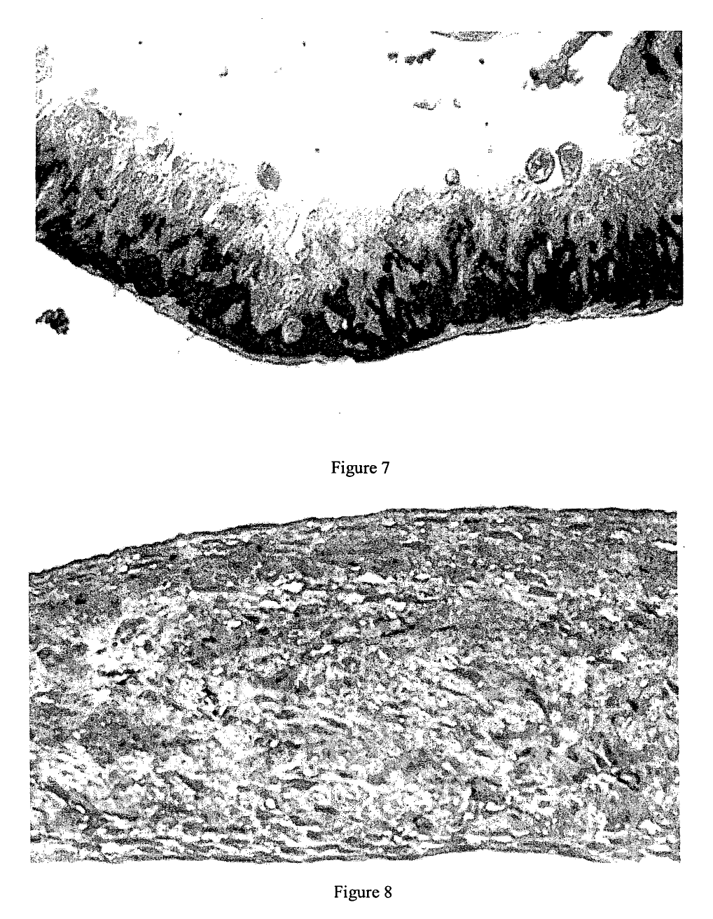

At the end of the culture period for the cells sampled in Example 1, a sheet was obtained which was recovered by detachment. The construct is shown in FIGS. 1 to 5.

### Example 3

### Implantation of a Construct in vivo

This example describes the in vivo introduction of an implant of the invention comprising a tissue construct.

The construct was prepared as described in Examples 1 and 2 from human periodontal ligament cells (PDL), then rolled around a bioglass implant (45 S 5). The construct was stored in DMEM medium supplemented with an antibiotic (gentamicin). Athymic male mice (Swiss NU/NU, 4 weeks old) were anesthetized. A subcutaneous implant was inserted on the dorsal side. To do so, four pouches were prepared on each mouse under sterile conditions. The implants were inserted in these pouches, which were then sutured. The sutures were covered with a dressing.

One month post-implantation, the mice were sacrificed and the constructs were recovered with the adjacent tissue of murine origin. All this was resin-embedded after fixation. Sections were prepared and stained with paragon. The sections were analyzed, and the constructs obtained were compared with those produced from skin fibroblasts, treated similarly, and with constructs which were not implanted. The histological sections showed that the PDL-derived constructs according to the invention and implanted in the mice displayed dense fibrillar structures, similar to those found in native periodontal ligament. The results further show that all the fibers were anchored to the implant surface by a thick mineralization layer, of cellular origin. Said tissue structures were absent from the other preparations.

These results highlight the advantages of the inventive tissues and methods, allowing the production of constructs having advantageous biological properties and compatible with use in vivo. Implantation in vivo of the constructs of the invention stimulates the recruitment of factors exogenous to the source biopsy used to prepare the construct, and leads to a maturation of the construct which contains biologically active cells.

### Example 4

### “Ligaplant” Preparation from Autolocous Cells

The “Ligaplant” is a product designed to replace a lost tooth in a patient, associating a ligament reconstructed from cells of said patient, and an implant produced from a biomaterial. The periodontal ligament is a ligament anchored on the root, principally, by cellular cements and acellular fibrils. Said ligament is also attached to the alveolar bone by a fibrillar structure embedded in the bone (Sharpey fibers). The ligament is reconstructed at the implant surface, by uniting the conditions for in vitro synthesis of an anchor similar to cement, before reimplanting the product. Restoring the anchor on the alveolar side is done once the product is reimplanted. The presence of this newly formed tissue structure orients the healing process, by avoiding osteointegration of the implant. The use of the patient's cells to reconstruct the ligament part of the Ligaplant makes it an autologous product.

A heterogeneous population was extracted from the surface of the lost tooth by scraping the median part of the root. Said population comprised the main cell types of the PDL: fibroblasts, cementoblasts, endothelial cells, and progenitors of these different cell types. The presence of these cells was confirmed both by examination under a light microscope, but also by the results of molecular biological analyses carried out on this population derived directly from the root surface biopsy. RT-PCR detection of messenger RNA coding for alkaline phosphatase, osteocalcin (OCN), osteopontin (OPN), bone sialo-protein (BSP) and type I collagen, confirmed both the heterogeneity of the population by the diversity of the cellular markers expressed, but also the possibilities, which exist, to reconstruct the PDL from said cells. During the proliferation phase conducted during the product manufacturing process, high-level synthesis of type I collagen demonstrating the presence of active fibroblasts in the culture was observed. At the same time, alkaline phosphatase and specific markers of cells capable of mineralizing the tissue structure (OPN, OCN, BSP) were expressed.

These specific activities were conserved throughout the process and were exploited to constitute the tissue part of the Ligaplant.

The biopsy from the root surface of the lost tooth was treated with collagenase, so as to accelerate extraction of the cells. A study carried out by the inventors demonstrated that the use of collagenase did not reduce either the cells' proliferative capacities or their capacities to express specific functions (mineralization and extracellular matrix production). At the end of the extraction phase, the population was a heterogeneous population of cells with well defined morphology (round cells:cementoblasts, elongated cells:fibroblasts).

The cell population was first amplified to increase the number of harvested cells. Cell proliferation was maintained at a high level in the exponential phase of the growth curve by repeated subcultures, carried out before the culture reached confluence. During this phase, the culture conditions (composition of medium, culture technique) led to a selection of cells: endothelial cells disappeared, since they were unable to adhere to the support or proliferate, while fibroblasts actively multiplied. The presence of cementoblasts could be detected by the expression level of their specific mineralization markers. The difference in cementoblast morphology, which allowed their identification under the microscope, disappeared, indicating that their shape became more spindle-like similar to fibroblasts. This is a common phenomenon, observed during the culture of cells present in mineralized tissue structures, of the chondrocyte, osteocyte type. However, this dedifferentiation of cementoblasts seemed to be restricted to their morphology. In fact, the production of zones of mineralization, induced in vitro by addition of β-glycerophosphate to the culture medium, demonstrates that said cells were functional.

After the third passage, the cells were not detached but left in the Petri dish in the presence of proliferation medium, supplemented with vitamin C to stimulate synthesis of extracellular matrix. A solid carpet of cells formed after 3 weeks of culture, composed of several layers of cells imprisoned in a dense extracellular matrix. As soon as approximately 5.10⁵ cells per cm² of dish area were obtained, the culture was grown beyond confluence so as to induce high-level production of extracellular matrix. At the end of this step, the culture became structured and adopted the form of a thick sheet composed of cells inside a dense extracellular matrix.

This sheet was detached from the bottom of the dish by mechanical means (rolling), after which it could be manipulated, given its good mechanical properties. A 15 cm² piece of this sheet was cut with a scalpel. It was rolled in several layers around the implant (cylinder 5 mm in diameter and 11 mm in length) to constitute the cellular part of the “Ligaplant”.

The future “Ligaplant” was ready for the final phase of differentiation which must allow the cells to produce a mineral anchor. This phase was accomplished by culturing the construct in a mineralization-promoting medium for 2 weeks. Anchorage of the sheet on the implant was achieved by synthesis of a mineralization of extracellular matrix by the cells, in the presence of culture medium containing 10 mM β-glycerophosphate. This mineralization was guided by the nature of the cell-implant interface. Calcium phosphate ceramics may be used to obtain a direct attachment with a mineralizing tissue. The cells respond to contact with this biomaterial by producing a mineral anchor layer from which a cement is regenerated when the product is implanted in vivo.

To understand how such a cellular organization could differentiate in vivo, and how this primary anchor could evolve towards formation of a cement, the inventors implanted the sheet-implant constructs subcutaneously at a site on the skulls of nude mice (Swiss nu/nu). They then studied the kinetic profile of said constructs at 4, 8 and 12 weeks post-implantation, by varying different parameters such as site of the biopsy (apex, central part of root, neck), and type of implant material (dentin, hydroxyapatite, calcium phosphate-coated titanium).

The most significant results were obtained with a cell sheet composed of cells extracted from a PDL biopsy of the median part of the tooth root, applied on a hydroxyapatite implant. Through the use of immunolabeling (antibodies specific of human tissue components), the inventors checked that the constructs were not denatured by the presence of the surrounding murine tissue and that they conserved a structural identity specific of their origin. The labeling studies identified the origin of the reconstructed tissues after reimplantation. The inventors observed a well defined limit between the implant and its associated human cells and the surrounding murine tissue.

The extent of mineralization of the extracellular matrix was measured by suitable treatment of the histological sections. The results reveal a gradual change over time in the thickness of the mineralized layers present at the hydroxyapatite interface. As this process went along there appeared a marked development of collagen fibers embedded in this mineral substrate. The presence of cells included in the mineral part can be seen here and there. These structural elements were present over the entire cell-biomaterial interface. The conclusions of this histological analysis indicate that a tissue characteristic of fibrillar cell cement, with a large quantity of Sharpey fibers, was regenerated by PDL cells and anchored the sheet on the implant.

As the implant is subjected to significant stresses, it must be made of a material selected for optimal biocompatibility all while conserving a mechanical strength similar to that of “osteointegrated” implants. The material selected for the implant was produced by bioactivation of titanium.

The naturally produced apatite anchor layer is very thin (˜1 μm), which eliminates the risk of fragile rupture in the layer (71) and preserves the micro-roughness of the implant surface (and therefore the mechanical bond between the metal and mineral phases). In addition and due to the chemical and physiological method of titanium activation, the titanium-apatite bond is a strong covalent bond which constitutes a continuous transition between the two phases.

1-28. (Canceled)

29. Cell construct cultured in vitro, in the absence of exogenous matrix or scaffold, wherein said construct comprises (i) animal cells cultured in vitro in conditions ensuring formation of a three-dimensional tissue structure and (ii) an endogenous extracellular matrix in which said cells are imprisoned, and comprises, among said cells, cells having mineralization and/or ossification capacity.

30. Construct according to claim 29, wherein the thickness is comprised between 30 and 120 microns, preferably between 50 and 120 microns.

31. Construct according to claim 29, wherein the animal cells are mammalian cells, preferably human cells.

32. Construct according to claim 29, wherein animal cells are undifferentiated stem cells and/or cells derived from mature tissues of mammalian cells, preferably of human cells.

33. Construct according to claim 29, wherein the animal cells are derived from mature tissues selected in the group consisting of muscle, bone, tooth, cartilage, tendon and ligament, in particular periodontal ligament or anterior cruciate ligament.

34. Construct according to claim 29, wherein the animal cells are derived from the periodontal ligament and comprise fibroblasts, epithelial cells, cementoblasts, osteoblasts and/or progenitors of these different cell types.

35. Construct according to claim 29, wherein the animal cells are undifferentiated stem cells corresponding to mesenchymatous cells or cells isolated from a bone marrow extract.

36. Construct according to claim 29, wherein said construct comprises a single cell population or a combination of several cell populations.

37. Construct according to claim 29, wherein said construct comprises within its thickness a basal layer and an apical layer relative to the culture support, said basal layer being rich in collagen and additionally comprising fibroblasts and progenitor cells ensuring mineralization and/or ossification, and said apical layer being less rich in collagen than said basal layer.

38. Cell construct or tissue according to claim 29, wherein said construct or tissue is obtained by a method comprising culturing cells in vitro, in the absence of exogenous matrix or scaffold, in conditions ensuring formation of a construct comprising one or more layers of cells imprisoned in an endogenous extracellular matrix, and a step of detaching the construct from its culture support, folding or rolling the construct upon itself, so as to produce a reconstructed tissue having an increased thickness.

39. Cell construct or tissue according to claim 29, wherein said construct or tissue is obtained by a method comprising culturing in vitro, in the absence of exogenous matrix or scaffold, cells from the periodontal or anterior cruciate ligament in conditions ensuring formation of a construct comprising one or more layers of cells imprisoned in an endogenous extracellular matrix, and, preferably, a step of detaching the construct from its culture support, folding or rolling the construct upon itself, so as to produce a reconstructed tissue having an increased thickness.

40. Cell construct or tissue according to claim 29, wherein said construct or tissue is obtained by a method comprising an in vitro culture, in the absence of exogenous matrix or scaffold, in conditions ensuring formation of a construct comprising one or more layers of cells imprisoned in an endogenous extracellular matrix, and in conditions stimulating mineralization or ossification.

41. Cell construct cultured in vitro according to claim 29, wherein said construct comprises (i) animal cells cultured in vitro in conditions ensuring formation of a three-dimensional tissue structure and (ii) an endogenous extracellular matrix in which said cells are embedded (or imprisoned), and wherein it has a thickness greater than or equal to approximately 100 μm.

42. Cell construct according to claim 29, wherein said construct comprises (i) cells sampled from ligament, tendon, tooth and/or bone of mammal(s), cultured in vitro in conditions ensuring formation of a three-dimensional tissue structure and (ii) an endogenous extracellular matrix in which said cells are embedded (or imprisoned), and wherein it has a thickness greater than or equal to approximately 100 μm.

43. Cell construct according to claim 29, wherein said construct comprises (i) cells sampled from ligament, tendon, tooth and/or bone of mammal(s), cultured in vitro in conditions ensuring formation of a three-dimensional tissue structure and (ii) an endogenous extracellular matrix in which said cells are embedded (or imprisoned), in that it has a thickness greater than or equal to approximately 100 μm and wherein it contains cells having a mineralization capacity and/or mineralized zones.

44. Method for preparing a cell construct comprising (i) animal cells and (ii) an endogenous extracellular matrix in which said cells are imprisoned, and comprising, among said cells, cells having mineralization and/or ossification capacity, wherein said method comprises:
a) culturing the cells of interest in conditions adapted to synthesis of an extracellular matrix and to formation of a construct comprising one or more layers of cells imprisoned in the neosynthesized endogenous extracellular matrix, and
b) recovering the construct.

45. Method according to claim 44, wherein said method additionally comprises, prior to steps a) and b), the following steps a′) and b′):
a′) extracting the cells of interest from one or more tissues, and
b′) amplifying said extracted cells in a suitable medium, typically in the presence of ascorbic acid or a derivative of said acid.

46. Method according to claim 44, wherein said method additionally comprises a step c) of recovering the cell construct and a step d) of artificially increasing the thickness of said construct, in particular by folding, rolling the construct upon itself or retraction of the construct.

47. Method according to claim 44, wherein the cells are cultured in the presence of a stimulus selected in the group consisting of a mineralizing support or material, particles of said material, differentiation factors, conditioned medium, a synthetic substance and/or a natural substance.

48. Method according to claim 44, wherein the cells are cultured in the presence of a mineralizing support or material comprising hydroxyapatite, bioglass, a bone substitute, a ceramic, coral or a composite material the surface of which is coated with one of said compounds.

49. Method according to claim 44, wherein the cells are cultured in the presence of a mineralizing support or material produced from an inert material bioactivated by surface grafting of substances able to induce mineralization of the construct.

50. Method for preparing an implant, wherein said implant comprises a construct according to claim 29.

51. Method according to claim 50, wherein the implant is a dental or ligamental implant.

52. Method for preparing an implant by means of a construct comprising (i) animal cells and (ii) an endogenous extracellular matrix in which said cells are imprisoned, and comprising, among said cells, cells having mineralization and/or ossification capacity, wherein said method comprises rolling said construct at the implant surface and culturing said implant associated with said construct in conditions allowing to maintain or stimulate the proliferation and/or differentiation of the cells all while promoting the merging and remodeling of the different cell layers of said construct at the implant surface.

53. Method for evaluating in vitro molecules designed for therapeutic use, wherein said method implements a construct as defined in claim 29.

54. Kit for preparing a construct comprising (i) animal cells and (ii) an endogenous extracellular matrix in which said cells are imprisoned, and comprising, among said cells, cells having mineralization and/or ossification capacity, comprising a container and culture reagents comprising a culture medium containing basic compounds required for the culture of animal cells or synthetic or recombinant functional equivalents, a mineralizing material and optionally ascorbic acid or a derivative thereof.

55. Pharmaceutical composition, wherein said composition comprises a cell construct comprising (i) animal cells and (ii) an endogenous extracellular matrix in which said cells are imprisoned, and comprising, among said cells, cells having mineralization and/or ossification capacity, and a pharmaceutically acceptable vehicle.

56. Implant, wherein a part of said implant is covered with a cell construct comprising (i) animal cells and (ii) an endogenous extracellular matrix in which said cells are imprisoned, and comprising, among said cells, cells having mineralization and/or ossification capacity.

57. Implant, wherein said implant comprises a three-dimensional tissue structure constituted of an endogenous extracellular matrix produced by ligament, tooth, bone, tendon, cartilage, muscle or by a combination or mixture of several of said cell types, said cells being imprisoned in said matrix, said matrix further comprising a collagenic fibrillar structure anchored on the implant surface by a mineralized layer of cellular origin.

58. Implant according to claim 57, wherein said collagenic fibrillar structure essentially comprises Sharpey fibers.

59. Implant according to claim 57, wherein it is made of a biomaterial chosen among dentin, hydroxyapatite, calcium phosphate-coated titanium and bioactivated titanium.

60. Implant according to claim 57, wherein said collagenic fibrillar structure is present over the entire cell-biomaterial interface of said implant.

61. Implant according to claim 57, wherein said implant is a dental, a ligamental, a bone or a cartilage implant.

62. Implant according to claim 57, wherein said implant is a ligamental implant intended for repairing a ligament, in particular the anterior cruciate ligament.
